(19) United States
(12) Patent Application Publication
(10) Pub. No.: US 2026/0097026 A1
(43) Pub. Date: Apr. 9, 2026

(54) INHIBITION OF AORTIC VALVE CALCIFICATION

(71) Applicants: Arsenii Zabirnyk, Oslo (NO); Ingvar Jarle Vaage, Oslo (NO); Kåre-Olav Stensløkken, Oslo (NO)

(72) Inventors: Arsenii Zabirnyk, Oslo (NO); Ingvar Jarle Vaage, Oslo (NO); Kåre-Olav Stensløkken, Oslo (NO)

(21) Appl. No.: 19/115,899

(22) PCT Filed: Oct. 3, 2023
(86) PCT No.: PCT/IB2023/059929

§ 371 Date: Mar. 27, 2025

Related U.S. Application Data
(60) Provisional application No. 63/412,932, filed on Oct. 4, 2022.

(51) Int. Cl. A61K 31/4439; A61P 9/10

(52) U.S. Cl. CPC A61K 31/4439; A61P 9/10

(57) ABSTRACT

The present invention relates to compounds useful for inhibiting calcification of aortic valves and to the treatment of diseases including aortic sclerosis and stenosis, and in particular to the use of the proton pump inhibitors including prazole compounds for the aforesaid treatments.

### CROSS-REFERENCE TO RELATED APPLICATIONS

This application claims the benefit of U.S. Provisional Application No. 63/412,932, filed Oct. 4, 2022, the content of which is herein incorporated by reference in its entirety.

### FIELD OF THE INVENTION

The present invention relates to compounds useful for inhibiting calcification of aortic valves and to the treatment of diseases including aortic sclerosis and stenosis, and in particular to the use of the proton pump inhibitors rabeprazole for the aforesaid treatments.

### BACKGROUND OF THE INVENTION

Calcific aortic valve disease is a slowly progressive disorder that ranges from thickening of aortic valve leaflets (aortic sclerosis), to severe calcification with obstruction of blood flow (aortic stenosis).

Aortic valve stenosis is the third leading cardiovascular disease and it is the most common form of valvular heart disease. In Norway, data indicates that many patients that have heart surgery have early aortic stenosis without symptoms. The occurrence of aortic stenosis increases exponentially after 60 years of age. In Western Europe and North America, about 20% of the male population begins to have early changes at the age of 50, increasing to 50% at the age of 75.

At the present time, aortic stenosis is treated by valve replacement with biological or mechanical valves. The biological valves are placed by catheters, last 8-15 years, and do not require anticoagulation. Placement of mechanical valves requires open heart surgery and lifelong anticoagulation therapy, and last for around 20 years. After the start of symptoms, 50% of patients die within two years unless they undergo valve replacement.

What is needed in the art are drug therapies for preventing the progression of calcification in aortic valves.

### SUMMARY OF THE INVENTION

The present invention relates to compounds useful for inhibiting calcification of aortic valves and to the treatment of diseases including aortic sclerosis and stenosis, and in particular to the repurposed use of the proton pump inhibitors, especially prazole compounds, for the aforesaid treatments.

In some preferred embodiments, the present invention provides a prazole compound for use in inhibiting or preventing a disease associated with calcification of the aorta in a subject. In some preferred embodiments, the present invention provides methods for the treatment, prevention, or inhibition of a disease associated with calcification of the aorta in a subject in need thereof comprising administering to the subject an effective amount of a prazole compound.

In some preferred embodiments, the prazole compound is a racemic mixture of S- and R-enantiomers of the prazole compound. In some preferred embodiments, the prazole compound is an S-enantiomer. In some preferred embodiments, the prazole compound is an R-enantiomer. In some preferred embodiments, the prazole compound is selected from the group consisting of rabeprazole, omeprazole, lansoprazole, rabeprazole, pantoprazole, and esomeprazole. In some preferred embodiments, the prazole compound is a rabeprazole compound. In some preferred embodiments, the prazole compound is a omeprazole compound. In some preferred embodiments, the prazole compound is a lansoprazole compound. In some preferred embodiments, the prazole compound is a pantoprazole compound. In some preferred embodiments, the prazole compound is an esomeprazole compound.

In some preferred embodiments, the disease associated with calcification of the aorta is aortic sclerosis.

In some preferred embodiments, the disease associated with calcification of the aorta is aortic stenosis.

In some preferred embodiments, the subject is at risk from developing a disease associated with calcification of the aorta.

In some preferred embodiments, the subject has asymptomatic changes in the aortic valve.

In some preferred embodiments, the subject has a biological valve prosthesis.

In some preferred embodiments, the subject has been diagnosed with rheumatic heart disease.

In some preferred embodiments, the subject has been diagnosed with peripheral vascular calcification.

In some preferred embodiments, the disease is congenital calcification.

In some preferred embodiments, the subject has been diagnosed with a pathological calcification disease in an area of the body other than the aorta.

In some preferred embodiments, administration of an effective amount of the prazole compound reduces the amount of calcification of the aorta of the subject.

In some preferred embodiments, the prazole compound has the structure:

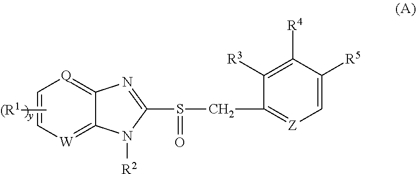

wherein
R1 is hydrogen, alkyl, halogen, cyano, carboxy, carboalkoxy, carboalkoxyalkyl, carbamoyl, carbamoylalkyl, hydroxy, alkoxy which is optionally fluorinated, hydroxyalkyl, trifluoromethyl, acyl, carbamoyloxy, nitro, acyloxy, aryl, aryloxy, alkylthio, or alkylsulfinyl; R2 is hydrogen, alkyl, acyl, acyloxy, alkoxy, amino, aralkyl, carboalkoxy, carbamoyl, alkylcarbamoyl, dialkylcarbamoyl, alkylcarbonylmethyl, alkoxycarbonylmethyl, or alkylsulfonyl;
R3 and R5 are the same or different and each is hydrogen, alkyl, C1-4 lower alkyl (e.g. methyl, ethyl, etc.), alkoxy, amino, or alkoxyalkoxy;
R4 is hydrogen, alkyl, C1-4 lower alkyl (e.g. methyl, ethyl, etc.), alkoxy which may optionally be fluorinated, or alkoxyalkoxy;
Q is nitrogen, CH, or CR1;
W is nitrogen, CH, or CR1;
y is an integer of 0 through 4; and
Z is nitrogen, CH, or CR1;
or a free base, salt, ester, hydrate, salt hydrate, amide, enantiomer, isomer, tautomer, prodrug, polymorph, or derivative thereof.

In some preferred embodiments, the prazole compound is provided as a pharmaceutical composition comprising an effective amount of the prazole compound and a pharmaceutically acceptable carrier or diluent.

In some preferred embodiments, the subject is at least 60 years of age. In some preferred embodiments, the subject is at least 75 years of age.

### BRIEF DESCRIPTION OF THE DRAWINGS

FIG. 1: Photomicrographs of control cells (no induced calcification, left panel), cells where calcification is induced (middle panel), and calcification in the presence of an inhibitor (right panel).

FIG. 2: Provides the results showing inhibition of calcification by a rabeprazole (denoted Rab in the figure) compound of the instant invention.

FIG. 3: Provides the results showing inhibition of calcification by three other FDA-approved proton pump inhibitors (prazoles) (denoted Class 1(omeprazole), Class 2 (pantoprazole) and Class 3 (lansoprazole)) in comparison to rabeprazole.

FIG. 4: Western blotting detection of proton pump alpha subunit (ATP4a) protein in human valves.

### DETAILED DESCRIPTION OF THE INVENTION

Aortic sclerosis and stenosis are serious conditions that lead to death is left untreated. The present invention provides therapies that can prevent calcification before it starts, or which can be used as soon as calcification begins (e.g., in asymptomatic patients).

In some preferred embodiments, the present invention provides a prazole compound for use in inhibiting or preventing a disease associated with calcification of the aorta in a subject. Prazole compounds of the present invention are H+, K+-ATPase proton pump inhibitors (PPIs). The term proton pump inhibitor or PPI means any pharmaceutical agent possessing pharmacological activity as an inhibitor of H+, K+-ATPase. Classes of PPIs include but are not limited to: substituted aryl-imidazoles, substituted bicyclic aryl-imidazoles, substituted benzimidazole compounds, and substituted imidazopyridines. A PPI useful in the present invention may, if desired, be in any form such as a free base, free acid, salt, ester, hydrate, anhydrate, salt hydrate, amide, enantiomer, isomer, tautomer, prodrug, polymorph, derivative, or the like, provided that the free base, free acid, salt, ester, hydrate, anhydrate, salt hydrate, amide, enantiomer, isomer, tautomer, prodrug, polymorph, or any other pharmacologically suitable derivative is therapeutically active or undergoes conversion within or outside of the body to a therapeutically active form.

In preferred embodiments, prazole compounds of use in the present invention are chemical entities having the following core structure:

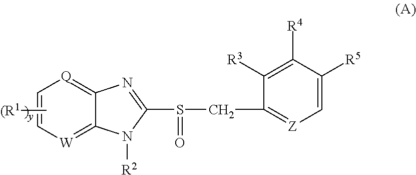

wherein
R1 is hydrogen, alkyl, halogen, cyano, carboxy, carboalkoxy, carboalkoxyalkyl, carbamoyl, carbamoylalkyl, hydroxy, alkoxy which is optionally fluorinated, hydroxyalkyl, trifluoromethyl, acyl, carbamoyloxy, nitro, acyloxy, aryl, aryloxy, alkylthio, or alkylsulfinyl;
R2 is hydrogen, alkyl, acyl, acyloxy, alkoxy, amino, aralkyl, carboalkoxy, carbamoyl, alkylcarbamoyl, dialkylcarbamoyl, alkylcarbonylmethyl, alkoxycarbonylmethyl, or alkylsulfonyl;
R3 and R5 are the same or different and each is hydrogen, alkyl, C1-4 lower alkyl (e.g. methyl, ethyl, etc.), alkoxy, amino, or alkoxyalkoxy;
R4 is hydrogen, alkyl, C1-4 lower alkyl (e.g. methyl, ethyl, etc.), alkoxy which may optionally be fluorinated, or alkoxyalkoxy;
Q is nitrogen, CH, or CR1;
W is nitrogen, CH, or CR1;
y is an integer of 0 through 4; and
Z is nitrogen, CH, or CR1;
or a free base, salt, ester, hydrate, salt hydrate, amide, enantiomer, isomer, tautomer, prodrug, polymorph, or derivative thereof. The “prazole compound,” can, if desired, be in any form such as a free base, free acid, salt, ester, hydrate, anhydrate, salt hydrate, amide, enantiomer, isomer, tautomer, prodrug, polymorph, derivative, or the like, provided that the free base, free acid, salt, ester, hydrate, anhydrate, salt hydrate, amide, enantiomer, isomer, tautomer, prodrug, polymorph, or any other pharmacologically suitable derivative is therapeutically active or undergoes conversion within or outside of the body to a therapeutically active form. The “prazole compound” can be either the R- or S-enantiomer, or a mixture thereof.

In some preferred embodiments, the disease associated with calcification of the aorta is aortic sclerosis. In some preferred embodiments, the disease associated with calcification of the aorta is aortic stenosis. Suitable prazoles include Class 1, Class 2 and Class 3 proton pump inhibitors such as rabeprazole, omeprazole, lansoprazole, rabeprazole, pantoprazole, and esomeprazole. See, e.g., U.S. Pat. No. 9,351,966, which is incorporated by reference herein in its entirety. In some preferred embodiments, the prazole compound is rabeprazole. In some preferred embodiments, the prazole compound is omeprazole. In some preferred embodiments, the prazole compound is pantoprazole. In some preferred embodiments, the prazole compound is lansoprazole. In some preferred embodiments, the prazole compound is esomeprazole. In some preferred embodiments, the prazole compound is dexlansoeprazole.

Prazoles are generally safe and well-tolerated drugs. Prazoles are currently used to treat the symptoms of gastroesophageal reflux disease (GERD), a condition in which backward flow of acid from the stomach causes heartburn and possible injury of the esophagus (the tube that connects the throat and stomach) in adults and children 1 year of age and older. Prazoles are used to treat damage from GERD, allow the esophagus to heal, and prevent further damage to the esophagus in adults. Prazoles are also used to treat conditions in which the stomach produces too much acid, such as Zollinger-Ellison syndrome in adults. Prazoles are used to treat ulcers (sores in the lining of the stomach or intestine) and is used in combination with other medications to eliminate H. pylori (a bacteria that causes ulcers) in adults.

In some preferred embodiments, the prazole compound is rabeprazole. Rabeprazole has the following structure:

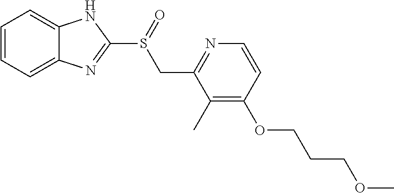 [SMILES: COCCCOc1ccnc(CS(=O)c2nc3ccccc3[nH]2)c1C]

In some preferred embodiments, the rabeprazole compound utilized in the present invention has the preceding structure. Rabeprazole compounds useful in the present invention are described in U.S. Pat. No. 5,054,552, incorporated herein by reference in its entirety. Additional rabeprazole compounds are described in WO1999/055157, U.S. Pat. Nos. 6,093,734, 6,093,734, 6,174,902, EP1073333, US200502341103, and WO2006/120701 all of which are incorporated herein by reference in their entirety. As used herein “rabeprazole compound” refers to chemical entities having structural homology to rabeprazole and the activity of a rabeprazole molecule. Examples include R-rabeprazole and S-rabeprazole molecules, rabeprazole prodrugs, and rabeprazole derivatives. In some preferred embodiments, the prazole compound is omeprazole. Omeprazole has the following structure:

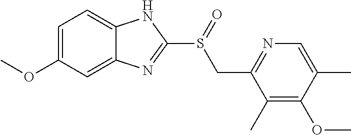

In some preferred embodiments, the omeprazole compound utilized in the present invention has the preceding structure. Omeprazole compounds useful in the present invention are described in EPO Pat. EP0005129B1 and U.S. Pat. Nos. 4,956,366, 5,075,323, 5,589,491, 5,690, 960, 6,162,816, 6,090,827 and 6,207,188, all of which are incorporated herein by reference in their entirety. As used herein “omeprazole compound” refers to chemical entities having structural homology to omeprazole and the activity of a omeprazole molecule. Examples include R-omeprazole and S-omeprazole molecules, omeprazole prodrugs, and omeprazole derivatives.

In some preferred embodiments, the prazole compound is pantoprazole. Pantoprazole has the following structure:

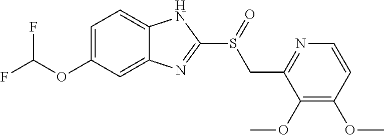 [SMILES: COc1ccnc(CS(=O)c2nc3cc(OC(F)F)ccc3[nH]2)c1OC]

In some preferred embodiments, the pantoprazole compound utilized in the present invention has the preceding structure. Pantoprazole compounds useful in the present invention are described in U.S. Pat. Nos. 4,758,579, 7,629,361 and 5,997,903, as wells as US20080234326 and WO2007029124, all of which are incorporated herein by reference in their entirety. As used herein “pantoprazole compound” refers to chemical entities having structural homology to pantoprazole and the activity of a pantoprazole molecule. Examples include R-pantoprazole and S-pantoprazole molecules, pantoprazole prodrugs, and pantoprazole derivatives.

In some preferred embodiments, the prazole compound is lansoprazole. Lansoprazole has the following structure:

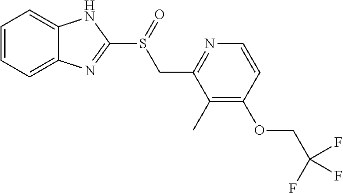 [SMILES: Cc1c(OCC(F)(F)F)ccnc1CS(=O)c1nc2ccccc2[nH]1]

In some preferred embodiments, the lansoprazole compound utilized in the present invention has the preceding structure. Lansoprazole compounds useful in the present invention are described in U.S. Pat. Nos. 4,628,098, 5,026,560, 6,909,004, and 7,285,668, as well as EP2535045 and WO20090324802, all of which are incorporated herein by reference in their entirety. As used herein “lansoprazole compound” refers to chemical entities having structural homology to lansoprazole and the activity of a lansoprazole molecule. Examples include R-lansoprazole and S-lansoprazole molecules, lansoprazole prodrugs, and lansoprazole derivatives.

In some preferred embodiments, the prazole compound is esomeprazole. Esomeprazole is the S-enantiomer of omeprazole. Esomeprazole has the following structure:

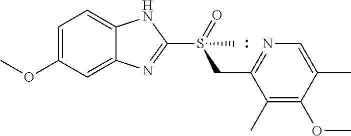

In some preferred embodiments, the esomeprazole compound utilized in the present invention has the preceding structure. Esomeprazole compounds useful in the present invention are described in U.S. Pat. Nos. 5,714,504, 7,563,812, and 8,063,074 as well as EP2106397B1, EP2143722A1, US20110213155, WO2007142580 and WO2006120520, all of which are incorporated herein by reference in their entirety. As used herein “esomeprazole compound” refers to chemical entities having structural homology to esomeprazole and the activity of a esomeprazole molecule. Examples include R-esomeprazole and S-esomeprazole molecules, esomeprazole prodrugs, and esomeprazole derivatives.

In some preferred embodiments, the prazole compound is dexlansoeprazole. Dexlansoprazole is the (R)-(+)-enantiomer of lansoprazole, which is a racemic mixture of its (R)-(+) and (S)-(−)-enantiomers. Dexlansoeprazole has the following structure:

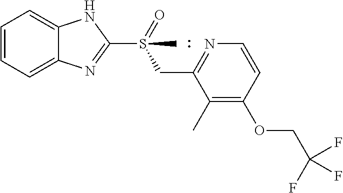

In some preferred embodiments, the dexlansoeprazole compound utilized in the present invention has the preceding structure. Dexlansoeprazole compounds useful in the present invention are described in U.S. Pat. Nos. 6,462,058 and 6,664,276, as well as US20090263475, all of which are incorporated herein by reference in their entirety. As used herein “dexlansoeprazole compound” refers to chemical entities having structural homology to dexlansoeprazole and the activity of a dexlansoeprazole molecule. Examples include R-dexlansoeprazole and S-dexlansoeprazole molecules, dexlansoeprazole prodrugs, and dexlansoeprazole derivatives.

Gastric acid inhibitors, including proton pump inhibitors as well as their salts, hydrates, esters, salt hydrates, amides, enantiomers, isomers, tautomers, polymorphs, prodrugs, and derivatives may be prepared using standard procedures that a person of ordinary skill in the art of synthetic organic chemistry would recognize. See, e.g., March, Advanced Organic Chemistry: Reactions, Mechanisms and Structure, 4th Ed. (New York: Wiley-Interscience, 1992); Leonard et al., Advanced Practical Organic Chemistry (1992); Howarth et al., Core Organic Chemistry (1998); and Weisermel et al., Industrial Organic Chemistry (2002).

“Pharmaceutically acceptable sals,” or “salts,” include the salt of a proton pump inhibitor prepared from formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic, methanesulfonic, ethanesulfonic, benzenesulfonic, pantothenic, toluenesulfonic, 2-hydroxyethanesulfonic, sulfanilic, cyclohexylaminosulfonic, algenic, beta-hydroxybutyric, galactaric and galacturonic acids.

In one embodiment, acid addition salts are prepared from the free base forms using, for example, methodologies involving reaction of the free base with a suitable acid. Suitable acids for preparing acid addition salts include both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like, as well as inorganic acids, e.g., hydrochloric acid, hydrobromic acid, sulfuric acid, nitric acid, phosphoric acid, and the like.

In other embodiments, an acid addition salt is reconverted to the free base by treatment with a suitable base. In a further embodiment, the acid addition salts of the proton pump inhibitors are halide salts, which are prepared, for example, using hydrochloric or hydrobromic acids. In still other embodiments, the basic salts are alkali metal salts, e.g., sodium salt. Salt forms of proton pump inhibitors include, but are not limited to: a sodium salt form such as esomeprazole sodium, omeprazole sodium, tenatoprazole sodium, rabeprazole sodium, pantoprazole sodium; a magnesium salt form such as esomeprazole magnesium or omeprazole magnesium, described in U.S. Pat. No. 5,900,424; a calcium salt form; a potassium salt form such as the potassium salt of esomeprazole, described in U.S. Pat. No. 6,511,996; salt hydrate forms including but not limited to sodium hydrate salt forms, for example tenatoprazole sodium hydrate or omeprazole sodium hydrate. Other salts of esomeprazole are described in U.S. Pat. Nos. 4,738,974 and 6,369,085. Salt forms of pantoprazole and lansoprazole are discussed in U.S. Pat. Nos. 4,758,579 and 4,628,098, respectively.

The foregoing list of suitable salts of proton pump inhibitors is meant to be illustrative and not exhaustive as a person of ordinary skill in the art would recognize that other pharmaceutically acceptable salts of a proton pump inhibitor could be created.

In one embodiment, preparation of esters involves functionalizing hydroxyl and/or carboxyl groups that may be present within the molecular structure of the drug. In another embodiment, the esters are acyl-substituted derivatives of free alcohol groups, e.g., moieties derived from carboxylic acids of the formula RCOOR₁, where R₁ is a lower alkyl group. Esters can be reconverted to the free acids, if desired, by using procedures including but not limited to hydrogenolysis or hydrolysis.

“Amides” may be prepared using techniques known to those skilled in the art or described in the pertinent literature. For example, amides may be prepared from esters, using suitable amine reactants, or they may be prepared from an anhydride or an acid chloride by reaction with an amine group such as ammonia or a lower alkyl amine.

“Tautomers” of substituted bicyclic aryl-imidazoles include, e.g., tautomers of omeprazole, such as those described in U.S. Pat. Nos. 6,262,085; 6,262,086; 6,268,385; 6,312,723; 6,316,020; 6,326,384; 6,369,087; and 6,444,689.

An exemplary “isomer” of a substituted bicyclic aryl-imidazole is the isomer of omeprazole including, but not limited to, isomers described in: Oishi et al., Acta Cryst. (1989), C45, 1921-1923; U.S. Pat. No. 6,150,380; U.S. Patent Publication No. 02/0156284; and PCT Publication No. WO 02/085889.

Exemplary “polymorphs” include, but are not limited to, those described in PCT Publication No. WO 92/08716, and U.S. Pat. Nos. 4,045,563; 4,182,766; 4,508,905; 4,628,098; 4,636,499; 4,689,333; 4,758,579; 4,783,974; 4,786,505; 4,808,596; 4,853,230; 5,026,560; 5,013,743; 5,035,899; 5,045,321; 5,045,552; 5,093,132; 5,093,342; 5,433,959; 5,464,632; 5,536,735; 5,576,025; 5,599,794; 5,629,305; 5,639,478; 5,690,960; 5,703,110; 5,705,517; 5,714,504; 5,731,006; 5,879,708; 5,900,424; 5,948,773; 5,997,903; 6,017,560; 6,123,962; 6,147,103; 6,150,380; 6,166,213; 6,191,148; 5,187,340; 6,268,385; 6,262,086; 6,262,085; 6,296,875; 6,316,020; 6,328,994; 6,326,384; 6,369,085; 6,369,087; 6,380,234; 6,428,810; 6,444,689; and 6,462,0577.

In one embodiment, at least one proton pump inhibitor is not enteric coated. In another embodiment, a portion of at least one proton pump inhibitor is optionally enteric coated. In another embodiment, a therapeutically effective portion of at least one proton pump inhibitor is optionally enteric coated. In another embodiment, about 5%, about 15%, about 20%, about 30%, about 40%, about 50% or about 60% of at least one proton pump inhibitor is optionally enteric coated. In another embodiment, a portion of at least one proton pump inhibitor comprises a “thin enteric coat.” The term “thin enteric coat” herein refers to a pH sensitive coating that is applied in a manner or amount such that it delays release of the coated substance in gastrointestinal fluid for a period of time, but ultimately allows release of some of the coated substance prior to passage into the duodenum.

In one embodiment, at least one proton pump inhibitor has a D90, D80, D70 or D50 particle size, by weight or by number, of less than about 900 μm, less than about 800 μm, less than about 700 μm, less than about 600 μm, less than about 500 μm, less than about 400 μm, less than about 300 μm, less than about 200 μm, less than about 150 μm, less than about 100 μm, less than about 80 μm, less than about 60 μm, less than about 40 μm, less than about 35 μm, less than about 30 μm, less than about 25 μm, less than about 20 μm, less than about 15 μm, less than about 10 μm, or less than about 5 μm.

In another embodiment, compositions are provided wherein a micronized proton pump inhibitor is of a size which allows greater than about 90%, greater than about 75%, or greater than about 50% of the proton pump inhibitor to be released from the dosage unit within about 1 hour, within about 50 minutes, within about 40 minutes, within about 30 minutes, within about 20 minutes, within about 10 minutes, or within about 5 minutes after placement in a standard dissolution test.

In still another embodiment, compositions of the disclosure comprise two PPIs in a total amount of about 1 mg to about 3000 μg, about 1 ng to about 2000 mg, about 1 mg to about 1000 μg, about 1 ng to about 750 mg, about 1 mg to about 500 mg, about 1 mg to about 300 mg, about 5 mg to about 250 mg, about 5 mg to about 200 μg, about 5 rug to about 175 μg, about 5 mg to about 120 mg, about 5 mug to about 100 mg, about 5 mg to about 80 mg, or about 5 mg to about 50 mg, for example about 5 mg, about 7.5 mg, about 10 mg, about 15 mg, about 20 mg, about 30 mg, about 40 mg, about 50 mg, about 60 nig, about 70 mg, about 80 mg, about 90 mg, about 100 mg, about 110 mg, about 120 mg, about 130 mug, about 140 mg, about 150 mug, about 160 mg, about 170 mg, about 180 mg, about 190 mg, or about 200 mg.

In still another embodiment, compositions of the disclosure comprise two PPIs where each is present in an amount of about 40 mg to about 160 mg, about 50 mg to about 150 mg, about 60 mg to about 140 mg, about 60 to about 130 mg, about 60 mg to about 120 mg, about 60 to about 110 mg, about 60 mg to about 100 mg, about 70 mg to about 100 mg, or about 80 to about 100 mg.

### EXAMPLES

### Example 1

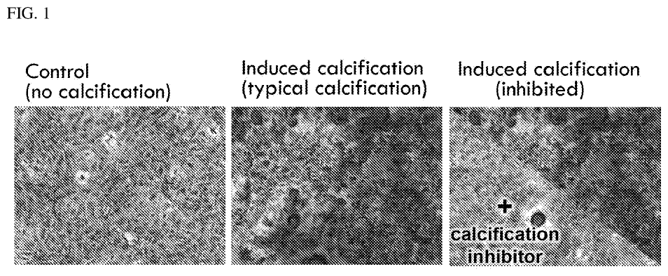

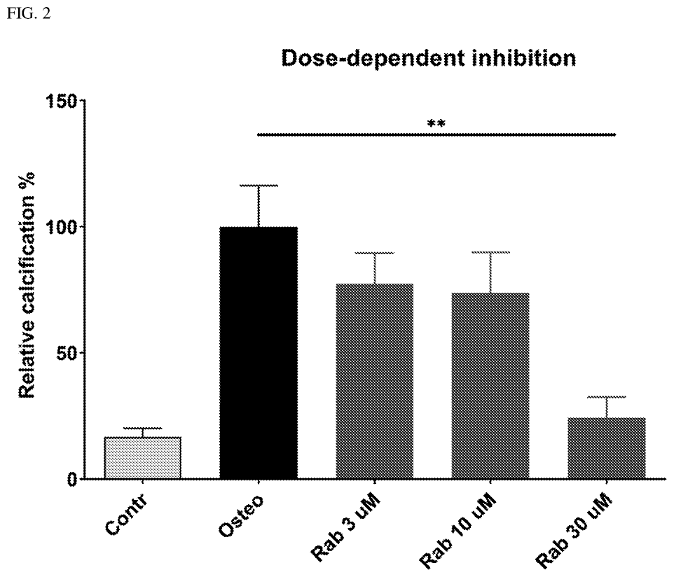

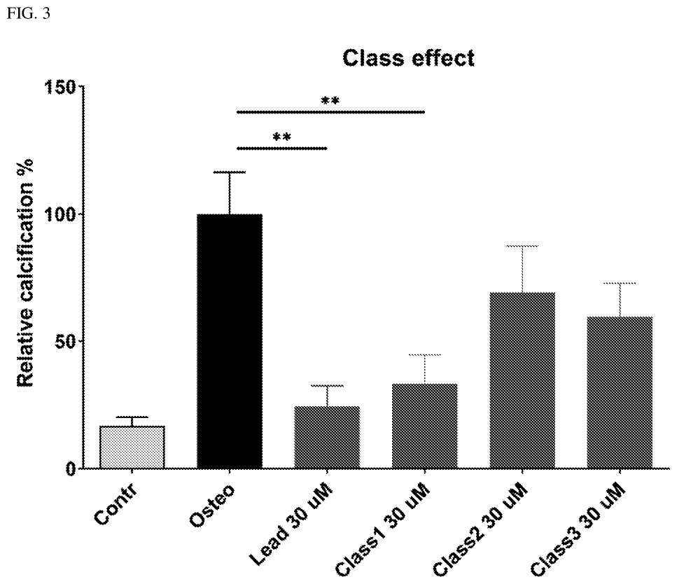

Human valve interstitial cells (VIC) are isolated from human aortic valves harvested during surgery for use in calcification inhibitor testing. After isolation, calcification is induced in the cells. The activity of potential inhibitors is assayed by contacting the VIC cells with the test substance during or after the induced calcification. The degree of calcification is measured by staining followed by spectroscopy. FIG. 1 provides photomicrographs of control cells (no induced calcification, left panel), cells where calcification is induced (middle panel), and calcification in the presence of an inhibitor (right panel). FIG. 2 provides the results showing inhibition of calcification by Rabeprazole denoted Rab in the figure). FIG. 3 Provides the results showing inhibition of calcification by three other FDA-approved proton pump inhibitors (prazoles) (denoted Class1, Class2 and Class3 in the figure) in comparison to rabeprazole.

### Example 2

This example provides data showing that aortic valve tissue contains proteins with sequence homology to the proton pump alpha subunit.

### Materials and Methods

In order to detect presence of proteins in aortic valve tissue with sequence homology to the proton pump alpha subunit (polypeptide responsible for covalent binding of prazole compounds), a crude protein extract was isolated from human aortic valves with protease inhibitor supplemented RIPA lysis buffer (Millipore) according to manufacturer's instructions. In a same way were prepared control rude protein extracts from rat tissue: stomach (positive control) and muscle, kidney (negative control). Protein detection was performed with Western blotting using Anti-ATP4A antibody (Abcam, EPR12251).

### Results:

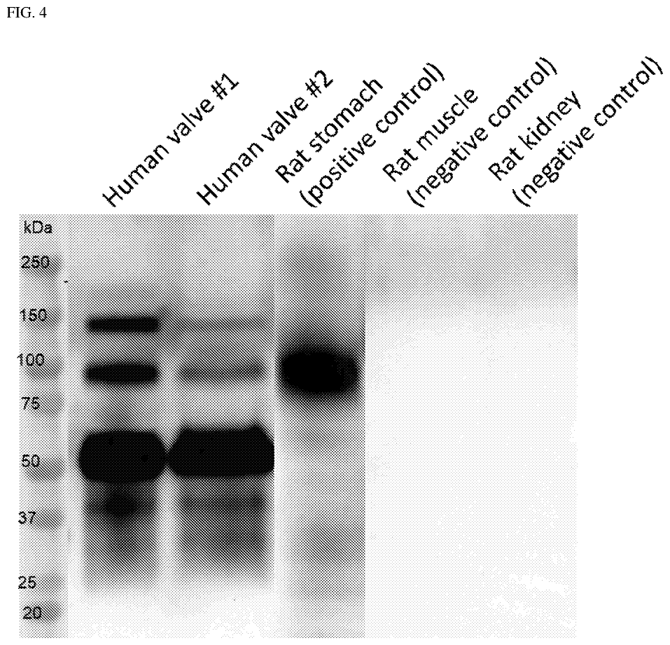

Four distinct proteins bands with apparent sequence homology to the human ATP4A protein were detected. (See FIG. 4). A single band with Mw of ˜95 kDa from valve tissue comigrated with the positive control from rat stomach, indicating the presence of the alpha subunit of the proton pump protein in aortic valve tissue.

A significant lower Mw band of ˜40, 55 kDa and a band at ˜140 kDa were also detected. The bands in human valve Western blots may indicate the presence of proton pump alpha subunits, protein conglomerates/subunits, cleavage, truncated forms or cross reactivity to proteins not related to the proton pump inhibitor. (Both the presence of protein with assumed homology to human proton pump alpha subunit and protein conglomerates/subunit cleavage polypeptides is novel and has not been investigated before.)

All publications, patents, patent applications and accession numbers mentioned in the above specification are herein incorporated by reference in their entirety. Although the invention has been described in connection with specific embodiments, it should be understood that the invention as claimed should not be unduly limited to such specific embodiments. Indeed, various modifications and variations of the described compositions and methods of the invention will be apparent to those of ordinary skill in the art and are intended to be within the scope of the following claims.

1-24. (canceled)

25. A method for the treatment, prevention, or inhibition of a disease associated with calcification of the aorta in a subject in need thereof comprising administering to the subject an effective amount of a prazole compound.

26. Method of claim 25, wherein the prazole compound is a racemic mixture of S- and R-enantiomers of the prazole compound.

27. Method of claim 25, wherein the prazole compound is an S-enantiomer.

28. Method of claim 25, wherein the prazole compound is an R-enantiomer.

29. Method of claim 25, wherein said prazole compound is selected from the group consisting of rabeprazole, omeprazole, lansoprazole, rabeprazole, pantoprazole, and esomeprazole.

30. Method of claim 25, wherein the prazole compound is a rabeprazole compound.

31. Method of claim 25, wherein the prazole compound is a omeprazole compound.

32-34. (canceled)

35. Method of claim 25, wherein the disease associated with calcification of the aorta is aortic sclerosis.

36. Method of claim 25, wherein the disease associated with calcification of the aorta is aortic stenosis.

37. Method of claim 25, wherein the subject is at risk from developing a disease associated with calcification of the aorta.

38. Method of claim 25, wherein the subject has asymptomatic changes in the aortic valve.

39. Method of claim 25, wherein the subject has a biological valve prosthesis.

40. Method of claim 25, wherein the subject has been diagnosed with rheumatic heart disease.

41. Method of claim 25, wherein the subject has been diagnosed with peripheral vascular calcification.

42. Method of claim 25, wherein the subject has congenital calcification.

43. Method of claim 25, wherein the subject has been diagnosed with a pathological calcification disease in an area of the body other than the aorta.

44. Method of claim 25, wherein administration of an effective amount of a prazole compound reduces the amount of calcification of the aorta of the subject.

45. (canceled)

46. Method of claim 25, wherein the rabeprazole compound is provided as a pharmaceutical composition comprising an effective amount of the rabeprazole compound and a pharmaceutically acceptable carrier or diluent.

47. Method of claim 25, wherein the subject is at least 60 years of age.

48. Method of claim 25, wherein the subject is at least 75 years of age.
